(19) United States
(12) Patent Application Publication
(10) Pub. No.: US 2015/0133322 A1
(43) Pub. Date: May 14, 2015

(54) METHOD FOR THE DIAGNOSIS OF EARLY RHEUMATOID ARTHRITIS

(71) Applicants: Jean Roudier, Marseille (FR); Isabelle Auger, Marseille (FR)

(72) Inventors: Jean Roudier, Marseille (FR); Isabelle Auger, Marseille (FR)

(73) Assignee: INSERM (INSTITUT NATIONAL DE LA SANTE ET DE LA RECHERCHE MEDICALE, Paris (FR)

(21) Appl. No.: 14/117,398

(22) PCT Filed: May 11, 2012
(86) PCT No.: PCT/EP12/58780

§ 371 Date: Dec. 19, 2014

(30) Foreign Application Priority Data
May 13, 2011 (EP) 11305584.2

(51) Int. Cl. G01N 33/564

(52) U.S. Cl. CPC G01N 33/564; G01N 2800/102
USPC 506/9; 506/18

(57) ABSTRACT

The present invention relates to biomarkers that are specifically recognized by autoantibodies present in a biological of patients with Rheumatoid Arthritis (RA). The invention provides methods and kits using these biomarkers for the diagnosis of RA, in particular for the diagnosis of early RA.

### RELATED APPLICATION

The present application claims priority to European Patent Application No. EP 11 305 584, which was filed on May 13, 2011. The European patent application is incorporated herein by reference in its entirety.

### FIELD OF THE INVENTION

The present invention relates to biomarkers that are specifically recognized by autoantibodies present in a biological sample of patients with Rheumatoid Arthritis (RA). The invention provides methods and kits for using these biomarkers for the diagnosis of RA, in particular for the diagnosis of early RA.

### BACKGROUND OF THE INVENTION

Rheumatoid arthritis is a chronic autoimmune disease affecting approximately 1% of the world's population. It is characterized by inflammation and cellular proliferation in the synovial lining of joints that can ultimately result in cartilage and bone destruction, joint deformity and loss of mobility. Rheumatoid arthritis usually causes problems in several joints at the same time, often in a symmetric manner. Early rheumatoid arthritis tends to affect the smaller joints first, such as the joints in the wrists, hands, ankles and feet. As the disease progresses, joints of the shoulders, elbows, knees, hips, jaw and neck can also become involved. Unlike other arthritic conditions that only affect areas in or around joints, rheumatoid arthritis is a systemic disease which can cause inflammation in extra-articular tissues throughout the body including the skin, blood vessels, heart, lungs and muscles.

Rheumatoid arthritis is associated with pain, deformity, decreased quality of life, and disability, which in turn affect patients' ability to lead a normal and productive life. Recent studies have shown that five years after the onset of the disease, approximately one third of patients with rheumatoid arthritis are no longer able to work, and within ten years, half of the patients have substantial functional disability (A. Young et al., Rheumatology, 2007, 46: 350-357). Consequently, rheumatoid arthritis imposes an important economic burden on society. Considerable data also suggest that rheumatoid arthritis is associated with lowered life expectancy.

Although rheumatoid arthritis has been extensively studied, the etiology and pathogenesis of the disease remain incompletely understood. Factors that may increase the risk for rheumatoid arthritis include: sex of the individual (women are 3 to 4 times more likely than men to develop the disease); age (rheumatoid arthritis occurs more commonly between the ages of 40 and 60, although it can also strike children, teenagers and older adults); genetics (rheumatoid arthritis was found to be strongly associated with Major Histocompatibility Complex (MHC) antigen HLA-DR4—more specifically DRB1*0401 and DRB1*0404); and smoking (rheumatoid arthritis is about 4 times more common in smokers than non-smokers).

There is currently no reliable cure for rheumatoid arthritis. Treatment is essentially directed towards relieving pain, reducing inflammation, and stopping or slowing joint damage and bone destruction. The current therapeutic approach is to prescribe disease-modifying antirheumatic drugs (DMARDs) early in the condition, as rheumatoid arthritis patients treated early with such drugs have better outcomes, with greater preservation of function, less work disability, and smaller risk of premature death. Recent advances in the understanding of the pathophysiology of rheumatoid arthritis have led to the development of new DMARDs, called biological response modifiers. Biological DMARDs are designed to target and block the action of certain key cells or molecules, such as tumor necrosis factor-alpha (TNF-α), interleukin-1 (IL-1), T-cells, and B-cells, involved in the abnormal immune reaction associated with rheumatoid arthritis. In comparison with traditional DMARDs, the biological agents have a much more rapid onset of action and can offer better clinical response with effective long-term prevention of joint damage (J. K. D. de Vries-Bouwstra et al., Rheum. Dis. Clin. North Am., 2005, 31: 745-762).

Since irreversible joint destruction can be prevented by intervention at the early stages of the disease, early diagnosis of rheumatoid arthritis is important. However, definitive diagnosis of rheumatoid arthritis can be difficult. Immunologic tests that can be performed for the diagnosis of rheumatoid arthritis include, in particular, measurement of the levels of rheumatoid factor (RF), and anti-cyclic citrullinated peptide (anti-CCP) antibodies. Serological testing for RF is complicated by moderate sensitivity and specificity, and high rates of positivity in other chronic inflammatory and infectious diseases (T. Dorner et al., Curr. Opin. Rheumatol, 2004, 16: 246-253). Anti-CCP antibody testing is particularly useful in the diagnosis of rheumatoid arthritis, with high specificity, positivity early in the disease process, and ability to identify patients who are likely to have severe disease and irreversible damage. However, a negative result in anti-CCP antibody testing does not exclude rheumatoid arthritis.

Therefore, there is a need for new biological markers of rheumatoid arthritis. In particular, biomarkers that would allow reliable diagnosis and monitoring of the early stages of the disease and permit early intervention to potentially prevent pain, joint destruction and long-term disability, are highly desirable.

### SUMMARY OF THE INVENTION

The inventors have found new autoantibodies associated with an early stage of rheumatoid arthritis (RA) and have identified 9 biomarkers which can be used for the detection said autoantibodies. For four of these biomarkers, the inventors have also identified epitopes specifically recognized by the sera of RA patients.

Thus, in one aspect, the present invention provides an in vitro method for diagnosing rheumatoid arthritis in a subject, said method comprising the step of detecting in a biological sample obtained from the subject one or more autoantibodies recognizing one or more protein biomarkers selected from the group of proteins consisting of WIBG-protein, TPM2-protein, C17orf85-protein, TCP10L-protein, ZNF706-protein, MGC17403-protein, CCDC72-protein, ELOF1-protein and GABARAPL2-protein. Alternatively, the method may be carried out with fragments of these proteins.

In preferred embodiments, a plurality of protein biomarkers (i.e., four or more than four protein biomarkers) is used in the method of diagnosis. In other words, the method of the invention may comprise steps of: contacting the biological sample with a plurality of biomarkers for a time and under conditions allowing biomarker-antibody complexes to form between four, five, six, seven, eight or nine biomarker and autoantibodies present in the biological sample; and detecting any biomarker-antibody complex formed.

In particular embodiments, the method of diagnosis is performed using nine different biomarkers including the WIBG-protein, TPM2-protein, C17orf85-protein, TCP10L-protein, ZNF706-protein, MGC17403-protein, CCDC72-protein, ELOF1-protein and GABARAPL2-protein, analogues thereof and fragments thereof as described herein.

In other particular embodiments, the method of diagnosis is performed using the following four biomarkers: the WIBG-protein, TPM2-protein, ZNF706-protein, and GABARAPL2-protein.

In certain embodiments, the WIBG-protein is a peptide having an amino acid sequence comprising or consisting of a sequence selected from the group consisting of SEQ ID NO: 10, SEQ ID NO: 11, SEQ ID NO: 12 and SEQ ID NO: 13.

In certain embodiments, the TPM2-protein is a peptide having an amino acid sequence comprising or consisting of a sequence selected from the group consisting of SEQ ID NO: 14 and SEQ ID NO: 15.

In certain embodiments, the ZNF706-protein is a peptide having an amino acid sequence comprising or consisting of SEQ ID NO: 16, SEQ ID NO: 17, and SEQ ID NO: 18.

In certain embodiments, the GABARAPL2-protein is a peptide having an amino acid sequence comprising or consisting of SEQ ID NO: 19 and SEQ ID NO: 20.

In particular embodiments, the subject to be tested is CCP-negative or the biological sample to be tested is obtained from a CCP-negative subject.

In preferred embodiments, the method of the invention is performed to detect patient with RA at an early stage.

In the methods of diagnosis provided herein, the step of detecting any biomarker-antibody complex formed between a protein biomarker and an autoantibody present in the biological sample may be performed by any suitable method. In certain embodiments, the detection is by immunoassay.

In particular embodiments, the protein biomarker or biomarkers used in the diagnosis method is/are immobilized on a solid carrier or support.

Typically, the methods of diagnosis may further comprise measuring, in a biological sample obtained from the subject, the concentration of at least one marker selected from the group consisting of C-reactive protein, serum amyloid A, interleukin 6, S100 proteins, osteopontin, rheumatoid factor, matrix metalloprotease 1, matrix metalloprotease 3, hyaluronic acid, sCD14, angiogenesis markers, and products of bone, cartilage and synovium metabolism.

In another aspect, the present invention provides kits for the in vitro diagnosis of RA in a subject. These kits comprise: one, two, three, four, five, six, seven, eight or nine biomarker of the invention and at least one reagent for detecting a biomarker-antibody complex formed between the protein biomarker and an autoantibody present in the biological sample to be tested. In the kits, the at least one protein biomarker may be immobilized on a solid carrier or support, or alternatively, reagents may be included in the kit that can be used to immobilize the protein biomarker on a solid carrier or support. The kits may further comprise instructions for carrying out a method of diagnosis according to the present invention. In certain embodiments, the kit comprises at least nine biomarkers including WIBG, TPM2, C17orf85, TCP10L, ZNF706, MGC17403, CCDC72, ELOF1 and GABARAPL2-proteins, or fragments thereof. In other embodiments, the kit comprises the following four biomarkers: the WIBG-protein, TPM2-protein, ZNF706-protein, and GABARAPL2-protein.

In certain preferred embodiments, these kits further comprise at least one additional RA biomarker for detecting the presence of RA-specific autoantibodies, such as PAD4-peptides BRAF-peptides and calpastatin-peptides (See WO2010115745 which is incorporated herein by reference).

In yet another aspect, the present invention provides arrays for the diagnosis of RA in a subject. An array according to the invention comprises, attached to its surface, at least one protein biomarker of the invention. In particular, the invention provides an array for the diagnosis of RA in CCP-negative subjects, the array comprising, attached to its surface, at least nine protein biomarkers including the WIBG, TPM2, C17orf85, TCP10L, ZNF706, MGC17403, CCDC72, ELOF1 and GABARAPL2 proteins described herein. In certain embodiments, the array comprises, attached to its surface, the following four biomarkers: the WIBG-protein, TPM2-protein, ZNF706-protein, and GABARAPL2-protein.

In certain embodiments, an inventive array further comprises, attached to its surface, at least one additional RA biomarker for detecting the presence of RA-specific autoantibodies, such as antinuclear antibodies and anti-CCP antibodies.

In preferred embodiments, the array further comprises, attached to its surface, at least one additional RA biomarker for detecting the presence of RA-specific autoantibodies, such as PAD4-peptides BRAF-peptides and calpastatin-peptides (See WO2010115745 which is incorporated by reference).

These and other objects, advantages and features of the present invention will become apparent to those of ordinary skill in the art having read the following detailed description of the preferred embodiments.

### DEFINITIONS

Throughout the specification, several terms are employed that are defined in the following paragraphs.

As used herein, the term “subject” refers to a human or another mammal (e.g., primate, dog, cat, goat, horse, pig, mouse, rat, rabbit, and the like), that can be afflicted with RA. In a preferred embodiment of the present invention, the subject is a human being. In such embodiments, the subject is often referred to as an “individual”. The term “individual” does not denote a particular age, and thus encompasses children, teenagers, and adults.

The term “subject suspected of having RA” refers to a subject that presents one or more symptoms indicative of RA (e.g., pain, stiffness or swelling of joints), or that is screened for RA (e.g., during a physical examination). Alternatively or additionally, a subject suspected of having RA may have one or more risk factors (e.g., age, sex, family history, smoking, etc). The term encompasses subjects that have not been tested for RA as well as subjects that have received an initial diagnosis.

The term “biological sample” is used herein in its broadest sense. A biological sample is generally obtained from a subject. A sample may be of any biological tissue or fluid with which biomarkers of the present invention may be assayed. Frequently, a sample will be a “clinical sample”, i.e., a sample derived from a patient. Such samples include, but are not limited to, bodily fluids which may or may not contain cells, e.g., blood (e.g., whole blood, serum or plasma), synovial fluid, saliva, tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, or proteins extracted from the sample. Processing of a biological sample may involve one or more of: filtration, distillation, extraction, concentration, inactivation of interfering components, addition of reagents, and the like.

In a preferred embodiment of the invention, the biological sample is a serologic sample and is or is derived from whole blood, serum or plasma obtained from a subject.

The terms “normal” and “healthy” are used herein interchangeably. They refer to a subject that has not presented any RA symptoms, and that has not been diagnosed with RA or with cartilage or bone injury. Preferably, a normal subject is not on medication for RA and has not been diagnosed with any other disease (in particular an autoimmune inflammatory disease). In certain embodiments, normal subjects may have similar sex, age, and/or body mass index as compared with the subject from which the biological sample to be tested was obtained. The term “normal” is also used herein to qualify a sample obtained from a healthy subject.

In the context of the present invention, the term “control”, when used to characterize a subject, refers to a subject that is healthy or to a patient who has been diagnosed with a specific disease other than RA. The term “control sample” refers to one, or more than one, sample that has been obtained from a healthy subject or from a patient diagnosed with a disease other than RA.

The term “autoantibody”, as used herein, has meaning accepted in the art, and refers to an antibody that is produced by the immune system of a subject and that is directed against subject's own proteins. Autoantibodies may attack the body's own cells, tissues, and/or organs, causing inflammation and damage.

As used herein, the term “autoantigen” refers to an endogenous antigen, or an active fragment thereof, that stimulates the production of autoantibodies in a body of a subject, as in autoimmune reactions. The term also encompasses any substances that can form an antigen-antibody complex with autoantibodies present in a subject or in a biological sample obtained from a subject.

The terms “biomarker” and “marker” are used herein interchangeably. They refer to a substance that is a distinctive indicator of a biological process, biological event, and/or pathologic condition. In the context of the present invention, the term “biomarker of RA” encompasses WIBG, TPM2, C17orf85, TCP10L, ZNF706, MGC17403, CCDC72, ELOF1 and GABARAPL2 proteins provided herein which are specifically recognized by anti-WIBG autoantibodies, anti-TPM2 autoantibodies, anti-C17orf85 autoantibodies, anti-TCP10L autoantibodies, anti-ZNF706 autoantibodies, anti-MGC17403 autoantibodies, anti-CCDC72 autoantibodies, anti-ELOF1 autoantibodies and anti-GABARAPL2 autoantibodies present in a biological sample (e.g., blood sample) of a RA patient. In certain preferred embodiments, the biomarkers of the invention are proteins fragment of less than 20 amino acids. In more preferred embodiments, the biomarkers of the invention are proteins fragment of between 5 and 20 amino acids (i.e. 10 or 15 amino acids).

As used herein, the term “indicative of RA”, when applied to a process or event, refers to a process or event which is a diagnostic of RA, such that the process or event is found significantly more often in subjects with RA than in healthy subjects and/or in subjects suffering from a disease other than RA. The term “early stage of RA” refers to the stage of the disease that spans one year after the onset of symptoms.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation, or citrullination. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulfhydryl groups. Thus, the term “protein” (or its equivalent terms) is intended to include the amino acid sequence of the full-length native protein, or a fragment thereof, subject to those modifications that do not significantly change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The term “analog”, when used herein in reference to a protein or polypeptide, refers to a peptide that possesses a similar or identical function as the protein or polypeptide but need not necessarily comprise an amino acid sequence that is similar or identical to the amino acid sequence of the protein or polypeptide or a structure that is similar or identical to that of the protein or polypeptide. Preferably, in the context of the present invention, an analog has an amino acid sequence that is at least 80%, more preferably, at least about: 80%, 85%, 90%, 95%, 96%, 97%, 98% or 99%, identical to the amino acid sequence of the protein or polypeptide. In certain preferred embodiments, an analog of a peptide biomarker of the invention has an amino acid sequence that is at least 80% identical or at least 85% identical to the amino acid sequence of the peptide biomarker.

The term “homologous” (or “homology”), as used herein, is synonymous with the term “identity” and refers to the sequence similarity between two polypeptide molecules or between two nucleic acid molecule. When a position in both compared sequences is occupied by the same base or same amino acid residue, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

The terms “labeled”, “labeled with a detectable agent” and “labeled with a detectable moiety” are used herein interchangeably. These terms are used to specify that an entity (e.g., a WIBG-protein, a TPM2-protein, a C17orf85-protein, a TCP10L-protein, a ZNF706-protein, a MGC17403-protein, a CCDC72-protein, a ELOF1-protein or a GABARAPL2-protein) can be visualized, for example, following binding to another entity (e.g., an anti-WIBG autoantibody, TPM2 autoantibody, C17orf85 autoantibody, TCP10L autoantibody, ZNF706 autoantibody, MGC17403 autoantibody, CCDC72 autoantibody, ELOF1 autoantibody and GABARAPL2 autoantibody). Preferably, a detectable agent or moiety is selected such that it generates a signal which can be measured and whose intensity is related to the amount of bound entity. In array-based methods, a detectable agent or moiety is also preferably selected such that it generates a localized signal, thereby allowing spatial resolution of the signal from each spot on the array. Methods for labeling proteins and polypeptides are well-known in the art. Labeled polypeptides can be prepared by incorporation of or conjugation to a label, that is directly or indirectly detectable by spectroscopic, photochemical, biochemical, immunochemical, electrical, optical, or chemical means, or any other suitable means. Suitable detectable agents include, but are not limited to, various ligands, radionuclides, fluorescent dyes, chemiluminescent agents, microparticles, enzymes, colorimetric labels, magnetic labels, and haptens.

The terms “protein array” and “protein chip” are used herein interchangeably. They refer to a substrate surface on which different proteins or polypeptides are immobilized, in an ordered manner, at discrete spots on the substrate. Protein arrays may be used to identify protein/protein interactions (e.g., antigen/antibody interactions), to identify the substrates of enzymes, or to identify the targets of biologically active small molecules. The term “microarray” specifically refers to an array that is miniaturized so as to require microscopic examination for visual evaluation.

The terms “CCP-negative patient” and “CCP-negative subject” are used herein interchangeably. They refer to a subject whose serum contains no antibodies (or at least no detectable antibodies) directed against citrullinated proteins.

### DETAILED DESCRIPTION OF CERTAIN PREFERRED EMBODIMENTS

As mentioned above, the present invention provides biomarkers that can be used for detecting the presence of RA-specific autoantibodies in biological samples obtained from patients. These biomarkers are WIBG-protein, TPM2-protein, C17orf85-protein, TCP10L-protein, ZNF706-protein, MGC17403-protein, CCDC72-protein, ELOF1-protein and GABARAPL2-protein which respectively and specifically react with anti-WIBG autoantibodies, anti-TPM2 autoantibodies, anti-C17orf85 autoantibodies, anti-TCP10L autoantibodies, anti-ZNF706 autoantibodies, anti-MGC17403 autoantibodies, anti-CCDC72 autoantibodies, anti-ELOF1 autoantibodies and anti-GABARAPL2 autoantibodies present in the serum of RA patients.

Other known biomarkers can be used with the diagnostic method of the present invention are PAD4-peptides, BRAF-peptides and calpastatin-peptides (See WO2010115745 which is incorporated herein by reference).

The invention also provides methods for using these biomarkers in the diagnosis of RA.

### I—Polypeptide/Protein Biomarkers

The term “WIBG-protein”, refers to the protein named “within bgcn homolog (Drosophila)” or “partner of Y14 and mago isoform 1” and referenced in the examples as P13. The sequence of said protein may be found with the NCBI Reference: NM_{—}032345 or NP_{—}115721. Protein P13 identified as described herein has the following amino acid sequence:

SEQ ID N^{o}1:
MEAAGSPAATETGKYIASTQRPDGTWRKQRRVKEGYVPQEEVPVYENKYV
KFFKSKPELPPGLSPEATAPVTPSRPEGGEPGLSKTAKRNLKRKEKRRQQ
QEKGEAEALSRTLDKVSLEETAQLPSAPQGSRAAPTAASDQPDSAATTEK
AKKIKNLKKKLRQVEELQQRIQAGEVSQPSKEQLEKLARRRALEEELEDL
ELGL

To be understood broadly, the term “WIBG” refers to the protein and also to analogues and fragments of the protein. The term “WIBG fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a WIBG protein. In preferred embodiments of the present invention, a fragment of a WIBG biomarker of the invention comprises an amino acid sequence of at least 5 consecutive amino acid residues of the amino acid sequence of the peptide biomarker and is not the whole protein.

In certain embodiments, a WIBG fragment is a peptidic fragment of the WIBG-protein comprising or consisting of an amino acid sequence selected from SEQ ID NO: 10, SEQ ID NO: 11, SEQ ID NO: 12 and SEQ ID NO: 13.

The term “TPM2-protein”, refers to the protein named “Tropomyosin 2 (Beta)” and referenced in the examples as P3. The sequence of said protein may be found under the NCBI Reference: NM_{—}003289 or NP_{—}003280. Protein P3 identified as described herein has the following amino acid sequence:

SEQ ID N^{o}2:
MDAIKKKMQMLKLDKENAIDRAEQAEADKKQAEDRCKQLEEEQQALQKKL
KGTEDEVEKYSESVKEAQEKLEQAEKKATDAEADVASLNRRIQLVEEELD
RAQERLATALQKLEEAEKAADESERGMKVIENRAMKDEEKMELQEMQLKE
AKHIAEDSDRKYEEVARKLVILEGELERSEERAEVAESKCGDLEEELKIV
TNNLKSLEAQADKYSTKEDKYEEEIKLLEEKLKEAETRAEFAERSVAKLE
KTIDDLEDEVYAQKMKYKAISEELDNALNDITSL

To be understood broadly, the term “TPM2” refers to the protein and also to analogues and fragments of the protein. The term “TPM2 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, 200, or 250 amino acid residues) of the amino acid sequence of a TPM2 protein. In preferred embodiments of the present invention, a fragment of a TPM2 biomarker of the invention comprises an amino acid sequence of at least 5 consecutive amino acid residues of the amino acid sequence of the peptide biomarker and is not the whole protein.

In certain embodiments, a TPM2 fragment is a peptidic fragment of the TPM2-protein comprising or consisting of an amino acid sequence selected from SEQ ID NO: 14, and SEQ ID NO: 15.

The term “C17orf85-protein”, refers to the protein named “ELG PROTEIN isoform B” or “chromosome 17 open reading frame 85” and referenced in the examples as P7. The sequence of said protein may be found under the NCBI Reference: NM_{—}018553 or NP_{—}061023. Protein P7 identified as described herein has the following amino acid sequence:

SEQ ID N^{o}3:
MKYGNPNYGGMKGILSNSWKRRYHSRRIQRDVIKKRALIGDDVGLTSYKH
RHSGLVNVPEEPIEEEEEEEEEEEEEEEEDQDMDADDRVVVEYHEELPAL
KQPRERSASRRSSASSSDSDEMDYDLELKMISTPSPKKSMKMTMYADEVE
SQLKNIRNSMRADSVSSSNIKNRIGNKLPPEKFADVRHLLDEKRQHSRPR
PPVSSTKSDIRQRLGKRPHSPEKAFSSNPVVRREPSSDVHSRLGVPRQDS
KGLYADTREKKSGNLWTRLGSAPKTKEKNTKKVDHRAPGAEEDDSELQRA
WGALIKEKEQSRQKKSRLDNLPSLQIEVSRESSSGSEAES

To be understood broadly, the term “C17orf85” refers to the protein and also to analogues and fragments of the protein. The term “C17orf85 fragment”, refers to a peptide comprising an amino acid sequence of at least 5 amino acid residues (preferably, of at least about: 10, 15, 20, 25, 30, 40, 50, 60, 70, 80, 90, 100, 125, 150, 175, 200, or 250 amino acid residues) of the amino acid sequence of a C17orf85 protein. In preferred embodiments of the present invention, a fragment of a C17orf85 biomarker of the invention comprises an amino acid sequence of at least 5 consecutive amino acid residues of the amino acid sequence of the peptide biomarker and is not the whole protein.

The term “TCP10L-protein”, refers to the protein named “T complex mouse like” and referenced in the examples as P9. The sequence of said protein may be found under the NCBI Reference: NM_{—}144659 or NP_{—}653260. Protein P9 identified as described herein has the following amino acid sequence:

SEQ ID N^{o}4:
MLAGQLEARDPKEGTHPEDPCPGAGAVMEKTAVAAEVLTEDCNTGEMPPL
QQQIIRLHQELGRQKSLWADVHGKLRSHIDALREQNMELREKLRALQLQR
WKARKKSAASPHAGQESHTLALEPAFGKISPLSADEETIPKYAGHKNQSA
TLLGQRSSSNNSAPPKPMSLKIERISSWKTPPQENRDKNLSRRRQDRRAT
PTGRPTPCAERRGGV

To be understood broadly, the term “TCP10L” refers to the protein and also to analogues and fragments of the protein. The term “TCP10L fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a TCP10L protein. In preferred embodiments of the present invention, a fragment of a TCP10L biomarker of the invention comprises an amino acid sequence of at least 5 consecutive amino acid residues of the amino acid sequence of the peptide biomarker and is not the whole protein.

The term “ZNF706-protein”, refers to the protein named “Zinc Finger Protein 706” or “HSPC038 protein” and referenced in the examples as P4. The sequence of said protein may be found under the NCBI Reference: NM_{—}016096 or NP_{—}057180. Protein P4 identified as described herein has the following amino acid sequence:

SEQ ID N^{o}5:
MARGQQKIQSQQKNAKKQAGQKKKQGHDQKAAAKAALIYTCTVCRTQMPD
PKTFKQHFESKHPKTPLPPELADVQA

To be understood broadly, the term “ZNF706” refers to the protein and also to analogues and fragments of the protein. The term “ZNF706 fragment”, refers to a peptide comprising an amino acid sequence of at least 5 amino acid residues (preferably, of at least: 10, 15, 20, 25, 30, 40, 50, 60, or 70 amino acid residues) of the amino acid sequence of a ZNF706 protein. In preferred embodiments of the present invention, a fragment of a ZNF706 biomarker of the invention comprises an amino acid sequence of at least 5 consecutive amino acid residues of the amino acid sequence of the peptide biomarker and is not the whole protein.

In certain embodiments, a ZNF706 fragment is a peptidic fragment of the ZNF706-protein comprising or consisting of an amino acid sequence selected from SEQ ID NO: 16, SEQ ID NO: 17 and SEQ ID NO: 18.

The term “MGC17403-protein”, refers to the protein named “hypothetical protein MGC17403” OR 3), or “Transcription elongation factor A (SII) N-terminal and central domain containing (TCEANC)” and refers in the examples as P6. The sequence of said protein may be found under the NCBI Reference: NM_{—}152634 or NP_{—}689847. Protein P6 identified as described herein has the following amino acid sequence:

SEQ ID N^{o}6:
MSDKNQIAARASLIEQLMSKRNFEDLGNHLTELETIYVTKEHLQETDVVR
AVYRVLKNCPSVALKKKAKCLLSKWKAVYKQTHSKARNSPKLFPVRGNKE
ENSGPSHDPSQNETLGICSSNSLSSQDVAKLSEMIVPENRAIQLKPKEEH
FGDGDPESTGKRSSELLDPTTPMRTKCIELLYAALTSSSTDQPKADLWQN
FAREIEEHVFTLYSKNIKKYKTCIRSKVANLKNPRNSHLQQNLLSGTTSP
REFAEMTVMEMANKELKQLRASYTESCIQEHYLPQVIDGTQTNKIKCRRC
EKYNCKVTVIDRGTLFLPSWVRNSNPDEQMMTYVICNECGEQWYHSKWVC
W

To be understood broadly, the term “MGC17403” refers to the protein and also to analogues and fragments of the protein. The term “MGC17403 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a MGC17403 protein. In preferred embodiments of the present invention, a fragment of a MGC17403 biomarker of the invention comprises an amino acid sequence of at least 5 consecutive amino acid residues of the amino acid sequence of the peptide biomarker and is not the whole protein.

The term “CCDC72-protein”, refers to the protein named “Coiled-Coil Domain Containing 72” or “hypothetical protein HSPC016” and referenced in the examples as P5. The sequence of said protein may be found under the NCBI Reference: NM_{—}015933 or NP_{—}057017. Protein P5 identified as described herein has the following amino acid sequence:

SEQ ID N^{o}7:
MSGREGGKKKPLKQPKKQAKEMDEEDKAFKQKQKEEQKKLEELKAKAAGK
GPLATGGIKKSGKK

To be understood broadly, the term “CCDC72” refers to the protein and also to analogues and fragments of the protein. The term “CCDC72 fragment”, refers to a peptide comprising an amino acid sequence of at least 5 amino acid residues (preferably, of at least about: 10, 15, 20, 25, 30, 40, 50, or 60 amino acid residues) of the amino acid sequence of a CCDC72 protein. In certain preferred embodiments of the present invention, a fragment of a CCDC72 biomarker of the invention comprises an amino acid sequence of at least 5 consecutive amino acid residues of the amino acid sequence of the peptide biomarker and is not the whole protein.

The term “ELOF1-protein”, refers to the protein named “Elongation Factor 1 Homolog (S. Cerevisiae)” and refers in the examples as P10. The sequence of said protein may be found under the NCBI Reference: NM_{—}032377 or NP_{—}115753. Protein P10 identified as described herein has the following amino acid sequence:

SEQ ID N^{o}8:
MGRRKSKRKPPPKKKMTGTLETQFTCPFCNHEKSCDVKMDRARNTGVISC
TVCLEEFQTPITYLSEPVDVYSDWIDACEAANQ

To be understood broadly, the term “ELOF1” refers to the protein and also to analogues and fragments of the protein. The term “ELOF1 fragment”, refers to a peptide comprising an amino acid sequence of at least 5 amino acid residues (preferably, of at least t: 10, 15, 20, 25, 30, 40, 50, or 60 amino acid residues) of the amino acid sequence of a ELOF1 protein. In certain preferred embodiments of the present invention, a fragment of a ELOF1 biomarker of the invention comprises an amino acid sequence of at least 5 consecutive amino acid residues of the amino acid sequence of the peptide biomarker and is not the whole protein.

The term “GABARAPL2-protein”, refers to the protein named “GABA(A) receptor-associated protein-like 2” and referenced in the examples as P23. The sequence of said protein may be found under the NCBI Reference: NM_{—}007285.5 or NP_{—}009216.1. Protein P23 identified as described herein has the following amino acid sequence:

SEQ ID N^{o}9:
MKWMFKEDHSLEHRCVESAKIRAKYPDRVPVIVEKVSGSQIVDIDKRKYL
VPSDITVAQFMWIIRKRIQLPSEKAIFLFVDKTVPQSSLTMGQLYEKEKD
EDGFLYVAYSGENTFGF

To be understood broadly, the term “GABARAPL2” refers to the protein and also to analogues and fragments of the protein. The term “GABARAPL2 fragment”, refers to a peptide comprising an amino acid sequence of at least 5 amino acid residues (preferably, of at least: 10, 15, 20, 25, 30, 40, 50, 60, 70, 80, 90, 100, or 110 amino acid residues) of the amino acid sequence of a GABARAPL2 protein. In certain preferred embodiments of the present invention, a fragment of a GABARAPL2 biomarker of the invention comprises an amino acid sequence of at least 5 consecutive amino acid residues of the amino acid sequence of the peptide biomarker and is not the whole protein.

In certain embodiments, a GABARAPL2 fragment is a peptidic fragment of the GABARAPL2-protein comprising or consisting of an amino acid sequence selected from SEQ ID NO: 19 and SEQ ID NO: 20.

### Preparation of the Peptide Biomarkers

The polypeptide/protein biomarkers of the present invention may be prepared by any suitable method, including recombinant methods. Such methods, as described, for example, in “The Proteins” (Vol. II, 3rd Ed., H. Neurath et al. (Eds.), 1976, Academic Press: New York, N.Y., pp. 105-237) may also be used to synthesize the biomarkers of the invention.

In certain embodiments, a polypeptide/protein biomarker of the invention is provided which is immobilized onto a solid carrier or support (e.g., a bead or array). Methods for immobilizing polypeptide molecules onto a solid surface are known in the art. A polypeptide/protein may be immobilized by being either covalently or passively bound to the surface of a solid carrier or support. Examples of suitable carrier or support materials include, but are not limited to, agarose, cellulose, nitrocellulose, dextran, Sephadex, Sepharose, carboxymethyl cellulose, polyacrylamides, polystyrene, polyvinyl chloride, polypropylene, filter paper, magnetite, ion-exchange resin, glass, polyamine-methyl-vinyl-ether-maleic acid copolymer, amino acid copolymer, ethylene-maleic acid copolymer, nylon, silk, and the like. Immobilization of a polypeptide/protein biomarker on the surface of a solid carrier or support may involve crosslinking, covalent binding or physical adsorption, using methods well known in the art. The solid carrier or support may be in the form of a bead, a particle, a microplate well, an array, a cuvette, a tube, a membrane, or any other shape suitable for conducting a diagnostic method according to the invention (e.g., using an immunoassay).

In particular, the invention provides an array or protein array for the diagnosis of RA, comprising, immobilized to its surface, at least one peptide biomarker of the invention. Preferably, the array comprises more than one polypeptide/protein biomarker of the invention. The array may further comprise at least one additional biomarker of RA. Suitable biomarkers of RA include biomarkers allowing detection of the presence of antinuclear antibodies and/or CCP antibodies.

The present invention also provides a protein bead suspension array for the diagnosis of RA. This bead suspension array comprises a suspension of one or more identifiable distinct particles or beads, wherein each bead contains coding features relating to its size, color or fluorescence signature and wherein each bead is coated with a polypeptide/protein biomarker of the present invention. Examples of bead suspension arrays include thexMAP® bead suspension array (Luminex Corporation).

### II—Methods of Diagnosis

As mentioned above, the biomarkers disclosed herein is specifically recognized by the sera of RA patients at an early stage, and in a particular embodiment by the sera of CCP-negative RA patients.

Accordingly, the present invention provides methods for the diagnosis of RA in a subject. Such methods comprise contacting a biological sample obtained from the subject to be tested with one, two, three, four, five, six, seven, eight or nine biomarkers for a time and under conditions allowing a biomarker-antibody complex to form; and detecting the biomarker-antibody complexes formed.

The biomarker may be selected from the group consisting of WIBG, TPM2, C17orf85, TCP10L, ZNF706, MGC17403, CCDC72, ELOF1 and GABARAPL2.

The present invention also provides methods for the diagnosis of RA in a subject. Such methods comprise contacting a biological sample obtained from the subject to be tested with one, two, three, four, five, six, seven, eight or nine biomarkers for a time and under conditions allowing a biomarker-antibody complex to form; and detecting any biomarker-antibody formed. The biomarkers may be selected from the group consisting of WIBG, TPM2, C17orf85, TCP10L, ZNF706, MGC17403, CCDC72, ELOF1 and GABARAPL2.

In this method, the detection of a biomarker-antibody complex is indicative of early RA in the subject.

In other embodiments, more than three biomarkers are used in combination, for example 4, 5; 6, 7, 8 or 9 biomarkers. In preferred embodiments, the combinations of biomarkers contain at least WIBG, TPM2, C17orf85, TCP10L.

In certain preferred embodiments, 9 biomarkers are used, i.e., WIBG, TPM2, C17orf85, TCP10L, ZNF706, MGC17403, CCDC72, ELOF1 and GABARAPL2.

In other preferred embodiments, 4 biomarkers are used, i.e., WIBG, TPM2, ZNF706, and GABARAPL2.

### Biological Samples

The method of diagnosis of the present invention may be applied to any type of biological sample allowing one or more biomarkers to be assayed. Examples of suitable biological samples include, but are not limited to, whole blood, serum, plasma, saliva, and synovial fluid. Biological samples used in the practice of the invention may be fresh or frozen samples collected from a subject, or archival samples with known diagnosis, treatment and/or outcome history. Biological samples may be collected by any non-invasive means, such as, for example, by drawing blood from a subject, or using fine needle aspiration or needle biopsy. In certain embodiments, the biological sample is a serologic sample and is selected from the group consisting of whole blood, serum, plasma.

In preferred embodiments, the inventive methods are performed on the biological sample itself without, or with limited, processing of the sample.

However, alternatively, the inventive methods may be performed on a protein extract prepared from the biological sample. In this case, the protein extract preferably contains the total protein content. Methods of protein extraction are well known in the art (see, for example “Protein Methods”, D. M. Bollag et al., 2nd Ed., 1996, Wiley-Liss; “Protein Purification Methods: A Practical Approach”, E. L. Harris and S. Angal (Eds.), 1989; “Protein Purification Techniques: A Practical Approach”, S. Roe, 2nd Ed., 2001, Oxford University Press; “Principles and Reactions of Protein Extraction, Purification, and Characterization”, H. Ahmed, 2005, CRC Press: Boca Raton, Fla.). Various kits can be used to extract proteins from bodily fluids and tissues. Such kits are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation.

### Detection of Biomarker-Antibody Complexes

The diagnostic methods of the present invention involve detection of a biomarker-antigen complex formed between the protein biomarker and an autoantibody present in the biological sample tested. In the practice of the invention, detection of such a complex may be performed by any suitable method (see, for example, E. Harlow and A. Lane, “Antibodies: A Laboratories Manual”, 1988, Cold Spring Harbor Laboratory: Cold Spring Harbor, N.Y.).

For example, detection of a biomarker-antibody complex may be performed using an immunoassay. A wide range of immunoassay techniques is available, including radioimmunoassay, enzyme immunoassays (EIA), enzyme-linked immunosorbent assays (ELISA), and immunofluorescence immunoprecipitation. Immunoassays are well known in the art. Methods for carrying out such assays as well as practical applications and procedures are summarized in textbooks. Examples of such textbooks include P. Tijssen, In: Practice and theory of enzyme immunoassays, eds. R. H. Burdon and v. P. H. Knippenberg, Elsevier, Amsterdam (1990), pp. 221-278 and various volumes of Methods in Enzymology, Eds. S. P. Colowick et al., Academic Press, dealing with immunological detection methods, especially volumes 70, 73, 74, 84, 92 and 121. Immunoassays may be competitive or non-competitive.

For example, any of a number of variations of the sandwich assay technique may be used to perform an immunoassay. Briefly, in a typical sandwich assay applied to the detection of, for example, anti-WIBG autoantibodies according to the present invention, an unlabeled WIBG-protein/polypeptide biomarker is immobilized on a solid surface (as described above) and the biological sample to be tested is brought into contact with the bound biomarker for a time and under conditions allowing formation of a biomarker-antibody complex. Following incubation, an antibody that is labeled with a detectable moiety and that specifically recognizes antibodies from the species tested (e.g., an anti-human IgG for human subjects) is added and incubated under conditions allowing the formation of a ternary complex between any biomarker-bound autoantibody and the labeled antibody. Any unbound material is washed away, and the presence of any anti-WIBG autoantibody in the sample is determined by observation/detection of the signal directly or indirectly produced by the detectable moiety. Variations on this assay include an assay, in which both the biological sample and the labeled antibody are added simultaneously to the immobilized WIBG-protein/polypeptide biomarker.

The second antibody (i.e., the antibody added in a sandwich assay as described above) may be labeled with any detectable moiety, i.e., any entity which, by its chemical nature, provides an analytically identifiable signal allowing detection of the ternary complex, and consequently detection of the biomarker-antibody complex.

Detection may be either qualitative or quantitative. Methods for labeling biological molecules such as antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

The most commonly used detectable moieties in immunoassays are enzymes and fluorophores. In the case of an enzyme immunoassay (EIA or ELISA), an enzyme such as horseradish perodixase, glucose oxidase, beta-galactosidase, alkaline phosphatase, and the like, is conjugated to the second antibody, generally by means of glutaraldehyde or periodate. The substrates to be used with the specific enzymes are generally chosen for the production of a detectable color change, upon hydrolysis of the corresponding enzyme. In the case of immunofluorescence, the second antibody is chemically coupled to a fluorescent moiety without alteration of its binding capacity. After binding of the fluorescently labeled antibody to the biomarker-antibody complex and removal of any unbound material, the fluorescent signal generated by the fluorescent moiety is detected, and optionally quantified. Alternatively, the second antibody may be labeled with a radioisotope, a chemiluminescent moiety, or a bioluminescent moiety.

### RA Diagnosis

In the method of the present invention, detection of a biomarker-antibody complex is indicative of the presence of WIBG autoantibodies, TPM2 autoantibodies, C17orf85 autoantibodies, TCP10L autoantibodies, ZNF706 autoantibodies, MGC17403 autoantibodies, CCDC72 autoantibodies, ELOF1 autoantibodies and GABARAPL2 autoantibodies in the biological sample tested and is therefore indicative of RA in the subject from which the biological sample is obtained. Thus, the methods of the present invention may be used for the diagnosis of RA in patients. In particular, the method of the invention may be used for testing subjects suspected of having RA.

It will be appreciated by one skilled in the art that diagnosis of RA may be performed solely on the basis of the results obtained by a method provided herein. Alternatively, a physician may also consider other clinical or pathological parameters used in existing methods to diagnose RA. Thus, results obtained using methods of the present invention may be compared to and/or combined with results from other tests, assays or procedures performed for the diagnosis of RA. Such comparison and/or combination may help provide a more refine diagnosis.

For example, RA diagnosis methods of the present invention may be used in combination with ARA criteria (i.e., the 2010 American College of Rheumatology/European League against Rheumatism classification criteria of RA described in D. Aletahal et al., Arthritis Rheum., 9 Sep. 2010, Vol 62: 2569-2581: see Table 3).

Alternatively or additionally, results from RA diagnosis methods of the present invention may be used in combination with results from one or more assays that employ other RA biomarkers. Thus, in certain embodiments, diagnosis of RA may be based on results from a method of the invention and on results from one or more additional assays that use a different RA biomarker. For example, a panel of RA biomarkers may be tested either individually or simultaneously, e.g., using a chip or a bead-based array technology.

Examples of suitable RA biomarkers include, but are not limited to, CCP, C-reactive protein, serum amyloid A, interleukin 6 (IL6), S100 proteins, ostopontin, rheumatoid factor, matrix metalloprotease 1 (MMP-1), matrix metalloprotease 3 (MMP-3), hyaluronic acid, sCD14, angiogenesis markers (such as the vascular endothelial growth factor or VEGF), and products of bone, cartilage or synovium metabolism (such as pyridino line or its glycosylated form; deoxy-pyridinoline; cross-linked telopeptides; collagen neoepitopes; CS846; cartilage oligomeric matrix protein; cartilage intermediate layer protein; matrilins, chondromodulatins, osteocalcin, and the like).

In preferred embodiments, the method of the invention is used for the diagnosis of RA in CCP-negative patients. Indeed, as reported herein in the Examples section, the inventors have shown, that a method of the invention using at least one of the nine proteins biomarkers (WIBG-protein, TPM2-protein, C17orf85-protein, TCP10L-protein, ZNF706-protein, MGC17403-protein, CCDC72-protein, ELOF1-protein and GABARAPL2-protein) allows the diagnosis of RA in more than 60% of CCP-negative subjects (⅗ of anti CCP negative RA patients were positive for at least one identified protein).

### III—Kits

In another aspect, the present invention provides kits comprising materials useful for carrying out a diagnostic method according to the present invention. The diagnosis procedures provided herein may be performed by diagnostic laboratories, experimental laboratories, or practitioners. The invention provides kits that can be used in these different settings.

Materials and reagents for detecting WIBG autoantibodies, TPM2 autoantibodies, C17orf85 autoantibodies, TCP10L autoantibodies, ZNF706 autoantibodies, MGC17403 autoantibodies, CCDC72 autoantibodies, ELOF1 autoantibodies and GABARAPL2 autoantibodies or any combination thereof in a biological sample and/or for diagnosing RA, in particular early RA, in a subject according to the present invention may be assembled together in a kit. Each kit of the invention comprises at least one protein/polypeptide biomarker of the invention preferably in an amount that is suitable for detection of autoantibodies in a bio logical sample.

Thus, in certain embodiments, a kit of the invention comprises one, two, three, four, five, six, seven, eight or nine biomarkers that are selected from the group consisting of WIBG, TPM2, C17orf85, TCP10L, ZNF706, MGC17403, CCDC72, ELOF1 and GABARAPL2.

In yet another embodiment, a kit of the invention comprises at least four; five, seven, eight or nine biomarkers selected from the group consisting of: WIBG, TPM2, C, 17orf85, TCP10L, ZNF706, MGC17403, CCDC72, ELOF1 and GABARAPL2.

In a preferred embodiment, the kit of the invention contains at least WIBG, TPM2, C17orf85, TCP10L.

In another preferred embodiment, the kit comprises at least nine biomarkers: WIBG, TPM2, C17orf85, TCP10L, ZNF706, MGC17403, CCDC72, ELOF1 and GABARAPL2.

In yet another preferred embodiment, the kit comprises the four following biomarkers: WIBG, TPM2, ZNF706 and GABARAPL2.

The peptide biomarker(s) included in a kit may or may not be immobilized on the substrate surface (e.g., beads, array, and the like). Thus, in preferred embodiments, a kit of the invention includes an array for diagnosing RA as provided herein. Alternatively, a substrate surface may be included in a kit of the invention for immobilization of the peptide biomarkers.

A kit of the invention generally also comprises at least one reagent for the detection of a biomarker-antibody complex formed between the peptide biomarker included in the kit and an autoantibody present in a biological sample. Such a reagent may be, for example, a labelled antibody that specifically recognizes antibodies from the species tested (e.g., an anti-human IgG for human subjects), as described above.

Depending on the procedure, the kit may further comprise one or more of the following: extraction buffer and/or reagents, blocking buffer and/or reagents, immunodetection buffer and/or reagents, labeling buffer and/or reagents, and detection means. Protocols for using these buffers and reagents for performing different steps of the procedure may be included in the kit.

The different reagents included in the kit of the invention may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit comprises instructions for using its components for the diagnosis of RA, in particular early RA, in a subject according to a method of the invention. Instructions for using the kit according to methods of the invention may comprise instructions for processing the biological sample obtained from the subject and/or for performing the test, and/or instructions for interpreting the results. A kit may also contain a notice in the form prescribed by a governmental agency regulating the manufacture, use or sale of pharmaceuticals or biological products.

The invention will be further illustrated by the following examples. However, this example should not be interpreted in any way as limiting the scope of the present invention.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Identification of 9 Autoantigens Indicative of Early RA Introduction

To identify new autoantibodies in RA, the inventors selected sera from 20 RA patients with disease duration of less than one year, 19 RA patients with disease duration of more than five years and 23 controls to screen 8000 human protein arrays. 24 “new autoantigens” associated with RA patients with disease duration of less than 1 year were identified. To confirm the validity of the protein array detection, the inventors used the same proteins in ELISA assays. Among the 24 proteins associated with early RA patients, the inventors validated 9 proteins that were preferentially recognized by autoantibodies from early RA patients. Indeed, these proteins were recognized by 21% to 47% of RA patients with disease duration less than 1 year and less than 5% of controls.

### Patients & Methods

RA Patients' Sera for Protein Array.

The inventors studied 39 RA patients from the rheumatology unit at La Conception Hospital in Marseille. Twenty RA patients had a disease duration of less than one year and 19 of more than five years. All RA patients fulfilled the American College of Rheumatology 1987 revised criteria. Ethical approval was obtained for this study (DC2008-327). All participants gave informed consent.

Controls' Sera for Protein Array.

Controls consisted of 7 patients with spondylarthropathy (AS) and 2 patients with systemic lupus erythematosus (SLE) from the rheumatology unit at La Conception Hospital in Marseille, 4 patients with systemic sclerosis (SSc) from a national cohort (Hospitals Cochin, Saint Antoine, Saint Louis, Paris, Hospital Claude Huriez, Lille and Hospital La Conception, Marseille). Ten healthy controls were recruited among laboratory staff volunteers and bone marrow of volunteer was used. All participants gave informed consent.

Human Protein Array.

The inventors used the “ProtoArray” from Invitrogen. This human protein microarray contains 8268 human proteins. Two percent are proteases or peptidases, 2% are secreted proteins, 3% are transcription factors, 3% are involved in cell death, 11% in signal transduction, 13% in cell communication, 5% are protein kinases, 13% are nuclear proteins, 17% are membrane proteins, 31% are involved in metabolism (data from Protein Content list 4.0).

All proteins were expressed as GST fusion proteins, purified under native conditions, and spotted in duplicate on nitrocellulose-coated glass slides. The human protein collection was derived from the human Ultimate ORF Clone Collection (data from http://orf.invitrogen.com). Each clone was sequence-checked on GenBank. Every clone used to generate the human protein collection contained a human ORF cloned into a Gateway® vector. The protein of interest was expressed as a N-terminal GST-fusion protein using a baculovirus expression system. After verifying that each clone expressed a protein of the expected molecular weight by western blotting, proteins were expressed and purified, spotted on nitrocellulose-coated glass slides.

Serum Profiling Assays on Protein Arrays.

Slides were blocked with 1% BSA/PBST. Serum samples were added to arrays. After washing, anti-human IgG conjugated to Alexa Fluor® 647 dye was added. Arrays were washed and dried (Partnership, Evry, FRANCE).

Data Acquisition/Analysis.

Arrays were scanned with a GenePix® 4000B Fluorescent Scanner. Data were acquired with GenePix® Pro software and processed using ProtoArray™ Prospector 2.0.

Data Analysis.

To assess the quality of the autoantigens identified, a panel of values was calculated for each protein array including the Z-Score, the CIP value (Chebyshev's Inequality Precision Value) and the CV (coefficient of variation between duplicate spots). The Z-Score is the signal value for a given spot minus the mean signal value from all the human proteins on the array, divided by the standard deviation of the signal values for all the human proteins on the array. The CIP value evaluates the signal strength for a given spot relative to all the negative control spots and calculates the probability that the observed signal may come from the negative control distribution. The lower the CIP value, the higher the probability that the signal is not due to a random event. The CV evaluates the similarity between duplicates. The lower the CV, the higher the similarity between duplicates. A Z-Score>3.0, a CI P value <0.05 and a CV<0.5 define a positive spot.

RA Patients and Controls for ELISA.

RA patients were chosen from the Rheumatology Ward at Hospital La Conception, Marseille, France and from the Rheumatology Ward at Hospital Jean Minjoz, Besancon, France. These patients fulfilled the 1987 American College of Rheumatology revised criteria for RA. 90% of early RA were anti CCP positive. Patients with spondylarthropathy (AS) and psoriasis arthritis (PsA) were chosen from the Rheumatology Ward at Hospital La Conception, Marseille, France. Volunteers from the laboratory staff and the Marseille Blood Transfusion Center staff served as normal controls. Ethical approval was obtained for this study (DC2008-327). All participants gave informed consent.

Detection of Autoantibodies by ELISA.

Plates were coated overnight with each purified protein diluted in phosphate buffer saline (PBS), pH7.4. Plates were blocked with PBS containing 5% milk. Sera diluted to 1:100 in PBS were incubated for 3 hours. After washing with 0.1% Tween 20, peroxydase conjugated anti human IgG (Sigma, France) was added. Optical density was read at 405 nm. Background OD was obtained by adding each serum to a well without protein. Positive sera were defined by an OD value more than twice background OD.

Statistical analysis.

p-values were calculated using the Chi squareTest.

### Results

Autoantibody Pattern Associated with Patients with Early RA.

20 sera from RA patients with disease duration less than 1 year were selected and their reactivity pattern on protein arrays were compared. 19 sera from RA patients with disease duration more than 5 years and 23 sera from control groups were already screened (Auger et al., Annals of Rheumatic Diseases, 2009, 68:591-594). The control groups were composed of 7 patients with spondylarthropathy (AS), 2 patients with lupus (SLE), 4 patients with systemic sclerosis (SSc) and 10 healthy controls. The presence of autoantibodies bound to each protein was detected by anti human IgG antibody.

The number of proteins recognized by each RA patient with disease duration of more than 5 years and control was very similar. On average, RA sera bound 101 proteins, AS sera bound 112 proteins, SLE sera bound 91 proteins, SSc sera bound 103 proteins and healthy control sera bound 89 proteins.

On the other hand, RA sera with disease duration of less than 1 year bound 58 proteins. Among these proteins, 24 proteins (P1 to P24) associated with early RA were identified. These proteins were recognized by 30% to 60% of RA patients with disease duration of less than 1 year and by less than 10% of controls (Table 1). Only P2, P4, P9, P10, P13, P15, P20 were also recognised by RA patients with disease duration of more than 5 years.

Identification of Specific Autoantibodies Associated with Early RA.

To confirm the validity of the protein array detection, the same 24 proteins were used in ELISA assays. Sera from 47 RA patients with disease duration less than 1 year, 25 RA patients with disease duration of more than 5 years, 66 AS patients, 21 PsA patients and 38 healthy controls were tested.

P11, P16, P12, P1, P15, P8, P22, P24, P14, P18, P2, P21, P20, P17 and P19 were less specific for early RA, 5% to 15% of controls were positive for these proteins.

TABLE 1
Protein arrays analysis

| | | | | Percentage of positive sera | | |
|---|---|---|---|---|---|---|
| pro- | | protein | | RA < 1 | RA 5 > | |
| tein | protein name | abbreviation | reference | year (20) | years (19) | Controls(23) |
| P1 | IMMUNOGLOBULIN (CD79A) BINDING PROTEIN 1 | IGBP1 | NM_001551.1 | 60 | 0 | 0 |
| P2 | FK506 binding protein | FKBP3 | NM_002013.2 | 45 | 5 | 10 |
| P3 | TROPOMYOSIN 2 (BETA) | TPM2 | NM_003289.3 | 45 | 0 | 0 |
| P4 | ZINC FINGER PROTEIN 706 | ZNF706 | NM_016096.1 | 45 | 2 | 10 |
| P5 | COILED-COIL DOMAIN CONTAINING 72 | CCDC72 | NM_015933.1 | 40 | 0 | 0 |
| | Hypothetical protein MGC17403 (MGC17403), Transcription | MGC17403 | | | | |
| | elongation factor A (SII) N-terminal and central domain containing | | | | | |
| P6 | (TCEANC) | | NM_152634.1 | 40 | 0 | 0 |
| P7 | ELG PROTEIN | C17orf85 | NM_018553.1 | 40 | 0 | 0 |
| P8 | HYPOTHETICAL PROTEIN MGC11257 | C7orf50 | NM_032350.3 | 40 | 0 | 0 |
| P9 | T complex mouse like | TCP10L | NM_144659.1 | 40 | 5 | 5 |
| P10 | ELONGATION FACTOR 1 HOMOLOG (S. CEREVISIAE) | ELOF1 | NM_032377.2 | 40 | 5 | 0 |
| P11 | FGF12 | FGF12 | NM_004113.3 | 40 | 0 | 0 |
| P12 | SYNAPTOTAGMIN I | SYT1 | NM_005639.1 | 40 | 0 | 0 |
| P13 | WITHIN BGCN HOMOLOG (DROSOPHILA) | WIBG | NM_032345.1 | 40 | 10 | 0 |
| P14 | YY1 TRANSCRIPTION FACTOR | YY1 | NM_003403.3 | 40 | 0 | 0 |
| P15 | eucaryotic transl factor1A | EIF1AX | NM_001412.2 | 40 | 5 | 5 |
| | DOUBLECORTEX LISSENCEPHALY, X-LINKED | DCX | | | | |
| P16 | (DOUBLECORTIN) | | NM_178151.1 | 35 | 0 | 0 |
| P17 | LAMIN A/C | LMNA | BC033088.1 | 35 | 0 | 0 |
| P18 | TROPOMYOSIN 4 | TPM4 | BC002827.1 | 30 | 0 | 0 |
| | ACID (LEUCINE-RICH) NUCLEAR PHOSPHOPROTEIN 32 | ANP32E | | | | |
| P19 | FAMILY, MEMBER E | | NM_030920.1 | 30 | 0 | 0 |
| P20 | HYPOTHETICAL PROTEIN MGC20255 | CCDC97 | NM_052848.1 | 30 | 5 | 0 |
| P21 | SPANX-N3 PROTEIN | SPANXN3 | NM_001009609.1 | 30 | 0 | 0 |
| P22 | THYMIC STROMAL LYMPHOPOIETIN | TSLP | NM_138551.1 | 30 | 0 | 0 |
| P23 | GABA(A) RECEPTOR-ASSOCIATED PROTEIN-LIKE 2 | GABARAPL2 | NM_007285.5 | 30 | 0 | 0 |
| P24 | SCY1like | SCYL1 | BC009967.1 | 30 | 0 | 5 |

The inventors found that P13, P3, P7, P9, P4, P6, P5, P10 and P23 were more specific for early RA. These proteins were recognized by 21 to 47% of RA patients with disease duration of less than 1 year and less than 5% of controls (Table 2).

TABLE 2
Autoantigen validation

| | | | Percentage of positive sera | | | | |
|---|---|---|---|---|---|---|---|
| | | | RA < 1 | RA > 5 | | | |
| | | | year (47) | years (25) | AS (66) | PSA (21) | Healthy (38) |
| WIBG | NM_032345.1 | P13 | 47% | 8% | 0% | 0% | 0% |
| TPM2 | NM_0032893 | P3 | 40% | 0% | 3% | 0% | 0% |
| C17orf85 | NM_018553.1 | P7 | 36% | 0% | 4.50% | 0% | 0% |
| TCP10L | NM_144659.1 | P9 | 36% | 4% | 4.50% | 0% | 5% |
| ZNF706 | NM_016096.1 | P4 | 28% | 8% | 1.50% | 0% | 3% |
| MGC17403 | NM_152634.1 | P6 | 28% | 0% | 1.50% | 0% | 0% |
| CCDC72 | NM_015933.1 | P5 | 25% | 0% | 3% | 0% | 3% |
| ELOF1 | NM_032377.2 | P10 | 25% | 4% | 0% | 0% | 3% |
| GABARAPL2 | NM_007285.5 | P23 | 21% | 0% | 1.50% | 5% | 3% |

Of interest, autoantibodies to P13 were very specific for early RA. Indeed, 47% of RA patients' sera recognized P13 versus 0% of AS patients (p<10-7), 0% of PsA patients (p=0.0001) and 0% of healthy individuals (p<10-5).

70% of early RA patients were seropositive for at least one protein (Table 3). Finally, ⅗ of anti CCP negative RA patients were positive for at least one identified protein (Table 3). These autoantibodies had a potential added value for RA diagnostic.

### Conclusions

In this study, the present inventors report the identification of 9 new autoantibodies associated with early RA. The sensitivities of these autoantibodies ranged from 21% to 47% while specificities were 95% to 100%. These proteins allow early diagnosis of RA patients.

TABLE 3
Autoantibody pattern associated with early RA

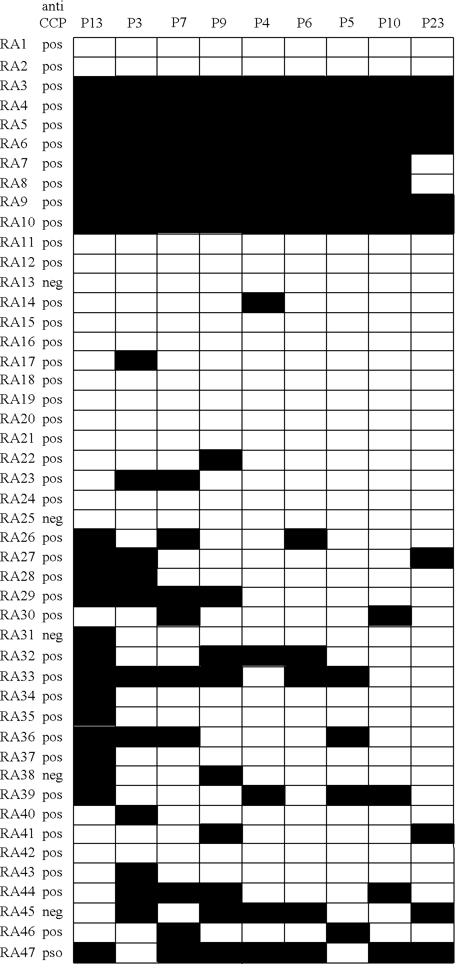

### Example 2

### Identification of 4 Autoantigens Indicative of Early RA Patients & Methods

See Example 1.

Serum Profiling Assays on Protein Arrays.

Protein arrays were processed at Partnerchip (Evry, France). Briefly, slides were blocked with phosphate buffer saline (PBS) containing 1% BSA (Bovine serum albumin). Serum samples diluted to 1:500 in PBS were added to arrays. After washing, anti-human IgG conjugated to Alexa Fluor® 647 dye was added. Arrays were scanned with a GenePix® 4000B Fluorescent Scanner. Data were acquired with GenePix® Pro software and processed using ProtoArray™ Prospector 2.0 (Invitrogen, Carlsbad, Calif., USA).

### Results

Autoantibodies Pattern Associated with RA Patients and Controls.

The inventors selected 20 sera from RA patients with disease duration of less than 1 year and compared their reactivity pattern on protein arrays with that of 19 sera from RA patients with disease duration of more than 5 years and 23 sera from controls. The control group included 7 patients with spondylarthropathy (AS), 2 patients with lupus (SLE), 4 patients with systemic sclerosis (SSc) and 10 healthy subjects. Autoantibodies were detected by anti human IgG antibody.

On average, the sera of RA patients with disease duration of more than 5 years were found to bind 101 proteins. AS sera bound 112 proteins, SLE sera bound 91 proteins, SSc sera bound 103 proteins and healthy controls' sera bound 89 proteins.

Sera from RA patients with disease duration of less than 1 year bound, on average, 58 proteins. Among these proteins, the inventors identified 25 proteins (P1 to P25) that were recognized by 30% to 60% of RA patients with disease duration of less than 1 year and less than 10% of controls (Table 4). These proteins were recognized by 0 to 37% of RA patients with disease duration more than 5 years.

TABLE 4
Protein array analysis

| | | | | Percentage of positive sera | | |
|---|---|---|---|---|---|---|
| pro- | | protein | | RA < 1 | Ra > 5 | |
| tein | protein name | abbreviation | reference | year (20) | years (19) | Controls (23) |
| P1 | IMMUNOGLOBULIN (CD79A) BINDING PROTEIN 1 | IGBP1 | NM_001551.1 | 60 | 0 | 0 |
| P2 | PEPTIDYL ARGININE DEIMINASE, TYPE IV | PAD4 | NM_012387.1 | 45 | 37 | 0 |
| P3 | FK506 binding protein | FKBP3 | NM_002013.2 | 45 | 5 | 10 |
| P4 | TROPOMYOSIN 2 (BETA) | TPM2 | NM_003289.3 | 45 | 0 | 0 |
| P5 | ZINC FINGER PROTEIN 706 | ZNF706 | NM_016096.1 | 45 | 2 | 10 |
| P6 | COILED-COIL DOMAIN CONTAINING 72 | CCDC72 | NM_015933.1 | 40 | 0 | 0 |
| P7 | Hypothetical protein MGC17403 (MGC17403), Transcription | MGC17403 | NM_152634.1 | 40 | 0 | 0 |
| | elongation factor A (SII) N-terminal and central domain | | | | | |
| | containing (TCEANC) | | | | | |
| P8 | ELG PROTEIN | C17orf85 | NM_018553.1 | 40 | 0 | 0 |
| P9 | HYPOTHETICAL PROTEIN MGC11257 | C7orf50 | NM_032350.3 | 40 | 0 | 0 |
| P10 | T complex mouse like | TCP10L | NM_144659.1 | 40 | 5 | 5 |
| P11 | ELONGATION FACTOR 1 HOMOLOG (S. CEREVISIAE) | ELOF1 | NM_032377.2 | 40 | 5 | 0 |
| P12 | FGF12 | FGF12 | NM_004113.3 | 40 | 0 | 0 |
| P13 | SYNAPTOTAGMIN I | SYT1 | NM_005639.1 | 40 | 0 | 0 |
| P14 | WITHIN BGCN HOMOLOG (DROSOPHILIA) | WIBG | NM_032345.1 | 40 | 10 | 0 |
| P15 | YY1 TRANSCRIPTION FACTOR | YY1 | NM_003403.3 | 40 | 0 | 0 |
| P16 | Eucaryotic transl factor 1A | EIF1AX | NM_001412.2 | 40 | 5 | 5 |
| P17 | DOUBLECORTEX LISSENCEPHALY, X-LINKED | DCX | NM_178151.1 | 35 | 0 | 0 |
| | (DOUBLECORTIN) | | | | | |
| P18 | LAMIN A/C | LMNA | BC033068.1 | 35 | 0 | 0 |
| P19 | TROPOMYOSIN 4 | TPM4 | BC002827.1 | 30 | 0 | 0 |
| | ACIDIC (LEUCINE-RICH) NUCLEAR PHOSPHOPROTEIN 32 | ANP32E | | | | |
| P20 | FAMILY, MEMBER E | | NM_030920.1 | 30 | 0 | 0 |
| P21 | HYPOTHETICAL PROTEIN MGC20255 | CCDC97 | NM_052848.1 | 30 | 5 | 0 |
| P22 | SPANX-N3 PROTEIN | SPANXN3 | NM_001009609.1 | 30 | 0 | 0 |
| P23 | THYMIC STROMAL LYMPHOPOIETIN | TSLP | NM_138551.1 | 30 | 0 | 0 |
| P24 | GABA(A) RECEPTOR-ASSOCIATED PROTEIN-LIKE 2 | GABARAPL2 | NM_007285.5 | 30 | 0 | 0 |
| P25 | SCY1like | SCYL1 | BC009967.1 | 30 | 0 | 5 |

Autoantibodies Specific for RA Patients.

To confirm the validity of protein array detection, we developed ELISA assays using purified proteins as immunosorbents. We tested sera from 68 RA patients with disease duration less than 1 year, 40 RA patients with disease duration more than 5 years, 76 AS patients, 27 PsA patients and 38 healthy subjects.

Eighteen (18) proteins targeted by autoantibodies from early RA were also recognized by autoantibodies from more than 5% of controls. These proteins are FGF12, DCX, SYT1, IGBP1, TCP10L, EIF1AX, C7orf50, C17orf85, TSLP, SCYL1, YY1, TPM4, FKBP3, SPANXN3, CCDC97, PAD4, LMNA and ANP32E.

7 proteins were recognized by more than 18% of early RA patients and less than 5% controls. These proteins are WIBG, TPM2, ELOF1, CCDC72, MGC17403, ZNF706 and GABARAPL2.

Specific Autoantibodies Associated with Early RA Patients.

WIBG, TPM2, ZNF706 and GABARAPL2 were recognized almost uniquely by sera from early RA patients (Table 5). Indeed, these proteins identified 18% to 41% of early RA patients and less than 5% of RA patients with disease duration more than 5 years or controls. Of interest, autoantibodies to WIBG were very specific for early RA. Indeed, 41% of RA patients' sera recognized WIBG vs 5% of RA patients with disease duration more than 5 years (p=0.00005), 0% of AS patients (p<10⁻⁷), 0% of PsA patients (p=0.0001) and 0% of healthy individuals (p<10⁻⁵).

TABLE 5
Validation of the early RA autoantigens

| | Percentage of positive sera | | | | | |
|---|---|---|---|---|---|---|
| | RA < 1 year | RA > 5 years | AS | PSA | Healthy | |
| | (68) | (40) | (76) | (27) | (38) | p |
| WIBG | 41% | 5% | 0% | 0% | 0% | p < 10⁻⁷ |
| TPM2 | 32% | 2.5% | 5% | 0% | 0% | p < 10⁻⁷ |
| ZNF706 | 25% | 5% | 1.3% | 0% | 3% | p < 10⁻⁷ |
| GABARAPL2 | 18% | 0% | 1.3% | 4% | 3% | p < 10⁻⁴ |

p value: early RA versus control groups (AS, PsA and healthy)

Moreover, 40 of 68 (60%) of early RA patients recognized at least one of these 4 proteins (Table 6).

TABLE 6
Binding of autoantibodies from early RA patients to WIBG, TPM2,
ZNF706 and GABARAPL2 by ELISA. Dark boxes indicate a positive
binding.

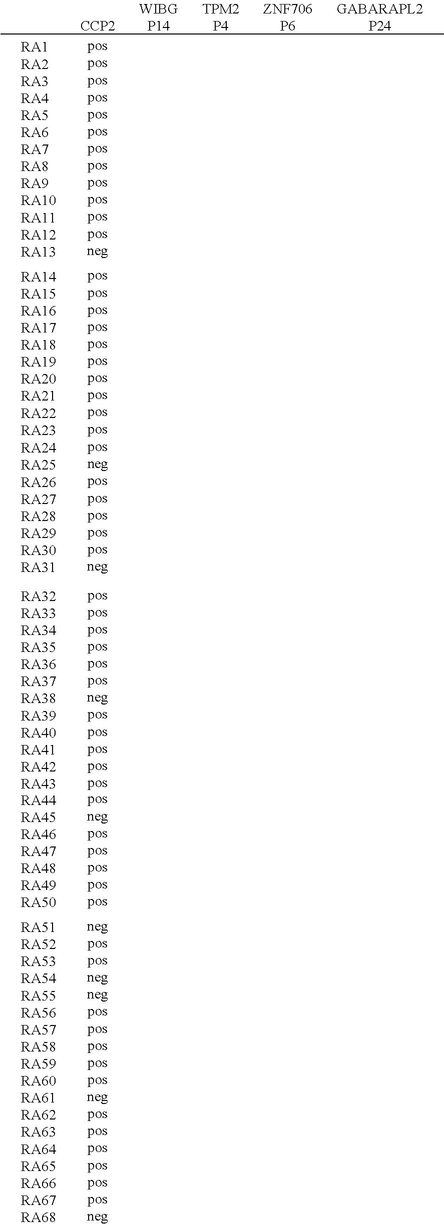

### Discussion

To get in insight in the early immunological events leading to the development of RA and to develop diagnosis tools for early RA, the inventors have screened 8000 protein arrays with sera from patients with early RA (less than one year), RA with disease duration of more than 5 years, ankylosing spondylitis, and lupus and from healthy subjects.

25 proteins that could be specific serologic markers of early RA were selected because they identified 30% to 60% of early RA patients and less than 10% of controls. Among these proteins, only one, PAD4, the enzyme that converts arginine into citrulline, recognized by more than 10% RA patients with disease duration of more than 5 years, was already known to be a target for RA autoantibodies.

To confirm the validity of the protein array detection, the same proteins were used in ELISA assays to screen more sera from patients and controls. To identify specific markers of early RA, all the proteins recognized by more than 5% of control groups and all the proteins recognized by RA patients with disease duration of more than 5 years were excluded. Four new autoantibody families associated with RA patients with disease duration of less than one year were identified. These autoantibodies recognize WIBG, TPM2, ZNF706 and GABARAPL2. These autoantibodies were recognized by 18% to 41% RA patients and less than 5% controls.

Autoantibodies to WIBG, TPM2, ZNF706 and GABARAPL2 were strongly associated with early RA. Indeed, autoantibodies to GABARAPL2 were found in early RA patients and not in RA patients with disease duration of more than 5 years. Autoantibodies to WIBG, TPM2 and ZNF706 were also found in RA patients with disease duration of more than 5 years but less often. The percentage of antibody positive RA patients decreased with disease duration. Indeed, WIBG was recognized by 41% of RA patients with disease duration of less than 1 year, 12% of RA patients with disease duration between 1 and 5 years and only 5% RA patients with disease duration of more than 5 years (data not shown).

Autoantibody decrease may be associated with treatment. Several studies have documented that the level of RA associated autoantibodies decreases with the administration of effective disease-modifying therapies. It is the case of anti-nuclear autoantibodies (ANA). ANA are positive in 20-30% of patients with RA. However, after the first few months of disease onset, ANA tests may turn negative and remain negative as disease develops (Goldbach-Mansky et al., Arthritis Res., 2000, 3: 236-243). This is also true for anti-Sa and rheumatoid factors. In treated recent-onset polyarthritis, anti CCP prevalence is stable or increases slightly, whereas anti-Sa and rheumatoid factor antibodies frequently disappear at 30 months (Guzian et al., Arthritis Care Res., 2010, 62: 1624-1632.

One year after the onset of the disease, 97% of RA patients have effective disease-modifying therapies. This could explain autoantibody decrease in RA patients with disease duration more than 5 years.

WIBG is a ribosome-associated protein involved in the disassembly of exon junction complexes (EJCs). EJCs, assembled during mRNA splicing, transport mRNAs during nuclear export into the cytoplasm and are removed during translation (Gehring et al., Cell, 2009, 137: 536-548). WIBG enhances translation of mRNAs (Diem et al., Nat. Struct. Mol. Biol., 2007, 14: 1173-1179). WIBG has also been shown to enhance the translation of viral genes, acting as a “chaperone” (Boyne et al., EMBO J., 2010, 29: 1851-1864).

WIBG (within BGCN homolog, Drosophila), might have diagnostic interest because of high sensitivity and specificity for early RA. Indeed, anti WIBG autoantibodies were detected in 41% of early RA patients and 5% of patients with RA with disease duration of more than 5 years, while these autoantibodies were not present in any control. Anti WIBG antibodies were detected in 26 of 58 (45%) anti CCP positive early RA patients and in 3 of 10 (30%) anti CCP negative early RA patients (Table 6). Together, these results indicate that WIBG could be used to diagnose patients with early RA.

TPM2 (tropomyosin 2) is a member of the actin filament binding protein family, involved in muscle contraction (Lin et al., Adv. Exp. Med. Biol., 2008, 644: 201-222).

ZNF706 (zinc finger protein 706) belongs to the C2H2-type zinc-finger protein family. A zinc finger protein is a DNA-binding protein whose specificity depends on its DNA-binding domain (Luchi et al., Cell. Mol. Life Sci., 2001, 58: 625-635; Laity et al., Curr. Opin. Struct. Biol., 2001, 11: 39-46).

GABARAPL2 (GABA(A) receptor associated protein like 2) is involved in autophagy, the process by which proteins and organelles are sequestered in autophagosomal vesicles and delivered to the lysosome for degradation (Diem et al., Nat. Stuct. Mol. Biol., 2007, 14: 1173-1179).

Autoantibodies to WIBG, TPM2, ZNF706 and GABARAPL2 can therefore be used to diagnose RA at an early stage of the disease.

### Example 3

### Epitope Mapping of 4 Autoantigens Indicative of Early RA

### Patients & Methods

As indicated in Example 2, four autoantigens (WIBG, TPM2, ZNF706 and GABARAPL2) were identified that are recognized almost uniquely by sera from early RA patients. To map B cell epitopes on these proteins, overlapping peptides encompassing each protein were analysed for their reactivity in RA sera by ELISA assays.

RA Patients' Sera for Peptide Screening.

RA patients with disease duration of less than one year were studied. All RA patients fulfil the American College of Rheumatology 1987 revised criteria. Ethical approval will be obtained for this study (DC2008-327). All participants give informed consent.

Controls' Sera for Peptide Screening.

Controls were patients with spondylarthropathy (AS), psoriasis arthritis (PsA), or systemic lupus erythematosus (SLE). Healthy controls were recruited among laboratory staff volunteers and volunteer bone marrow. All participants give informed consent (DC2008-327).

Synthetic Peptides.

15-mer peptides, overlapping by 7 amino acids for each biomarker were synthesized using the solid phase system (Eurogentec, France).

Detection of Autoantibodies by ELISA.

Plates were coated overnight with 10 μg/well of peptide diluted in phosphate buffer saline (PBS), pH7.4. Plates were blocked with PBS containing 5% milk. Sera diluted to 1:100 in PBS were incubated for 2 hours. After washing with 0.1% Tween 20, peroxydase conjugated anti human IgG, (Sigma, France) was added. Optical density was read at 405 nm. Background OD, was obtained by adding each serum to a well without peptide. A positive serum was defined as an OD value more than twice the background OD.

Statistical Analysis.

p-values are calculated using the Chi square Test.

### Results

Epitopes of WIBG.

The regions of the WIBG-protein that were found to be recognized by the sera of RA patients were:

SEQ ID NO: 10: MEAAGSPAATETGKY
SEQ ID NO: 11: AATETGKYIASTQRP
SEQ ID NO: 12: IASTQRPDGTWRKQR
SEQ ID NO: 13: KKKLRQVEELQQRIQ

Epitopes of TPM2.

The regions of the TPM2-protein that were found to be recognized by the sera of RA patients were:

SEQ ID NO: 14: QKMKYKAISEELDNA
SEQ ID NO: 15: ISEELDNALNDITSL

Epitopes of ZNF706.

The regions of the ZNF706-protein that were found to be recognized by the sera of RA patients were:

SEQ ID NO: 16: MARGQQKIQSQQKNA
SEQ ID NO: 17: IQSQQKNAKKQAGQK
SEQ ID NO: 18: KKQAGQKKKQGHDQK

Epitopes of GABARAPL2.

The regions of the GABARAPL2-protein that were found to be recognized by the sera of RA patients were:

SEQ ID NO: 19: MKWMFKEDHSLEHRC
SEQ ID NO: 20: GFLYVAYSGENTFGF.

What is claimed is:

1. A method for diagnosing rheumatoid arthritis in a subject, said method comprising the step of detecting in a biological sample obtained from the subject one or more autoantibodies recognizing one or more protein biomarkers selected from the group of proteins consisting of WIBG-protein, TPM2-protein, C17orf85-protein, TCP10L-protein, ZNF706-protein, MGC17403-protein, CCDC72-protein, ELOF1-protein, and GABARAPL2-protein.

2. The method according to claim 1, said method comprising steps of:
contacting a biological sample obtained from the subject with one, two, three, four, five, six, seven, eight or nine biomarkers selected from the group consisting of WIBG, TPM2, C17orf85, TCP10L, ZNF706, MGC17403, CCDC72, ELOF1, and GABARAPL2; and
detecting any biomarker-antibody complex formed,
wherein the detection of a biomarker-antibody complex is indicative of rheumatoid arthritis in the subject.

3. The method according to claim 2, wherein said biological sample obtained from the subject is in contact with 4, 5, 6, 7, 8 or 9 of said biomarkers.

4. The method according to claim 3, wherein said biological sample obtained from the subject is in contact with the 4 biomarkers: WIBG, TPM2, ZNF706, and GABARAPL2.

5. The method according to claim 3, wherein said biological sample obtained from the subject is in contact with said 9 biomarkers.

6. The method according to claim 1, wherein the WIBG-protein is a peptide having an amino acid sequence comprising, or consisting of a sequence selected from the group consisting of SEQ ID NO: 10, SEQ ID NO: 11, SEQ ID NO: 12 and SEQ ID NO: 13; the TPM2-protein is a peptide having an amino acid sequence comprising, or consisting of a sequence selected from the group consisting of SEQ ID NO: 14 and SEQ ID NO: 15; the ZNF706-protein is a peptide having an amino acid sequence comprising, or consisting of SEQ ID NO: 16, SEQ ID NO: 17, and SEQ ID NO: 18; and the GABARAPL2-protein is a peptide having an amino acid sequence comprising, or consisting of, SEQ ID NO: 19 and SEQ ID NO: 20.

7. The method according to claim 1, wherein the subject is CCP-negative.

8. The method according to claim 1, wherein the method is for diagnosing rheumatoid arthritis at an early stage.

9. A kit for the in vitro diagnosis of rheumatoid arthritis in a subject, said kit comprising:
one, two, three, four, five, six, seven, eight or nine biomarkers selected from the group consisting of WIBG, TPM2, C17orf85, TCP10L, ZNF706, MGC17403, CCDC72, ELOF1, and GABARAPL2; and
at least one reagent for detecting a biomarker-antibody complex formed between the biomarker and an autoantibody present in a biological sample obtained from the subject.

10. The kit according to claim 9, wherein said kit comprises said 9 biomarkers.

11. The kit according to claim 9, wherein said kit comprises the 4 biomarkers: WIBG, TPM2, ZNF706, and GABARAPL2.

12. The kit according to claim 9, wherein the WIBG-protein is a peptide having an amino acid sequence comprising, or consisting of, a sequence selected from the group consisting of SEQ ID NO: 10, SEQ ID NO: 11, SEQ ID NO: 12 and SEQ ID NO: 13; the TPM2-protein is a peptide having an amino acid sequence comprising, or consisting of a sequence selected from the group consisting of SEQ ID NO: 14 and SEQ ID NO: 15; the ZNF706-protein is a peptide having an amino acid sequence comprising, or consisting of, SEQ ID NO: 16, SEQ ID NO: 17, and SEQ ID NO: 18; and the GABARAPL2-protein is a peptide having an amino acid sequence comprising, or consisting of, SEQ ID NO: 19 and SEQ ID NO: 20.

13. An array for diagnosing rheumatoid arthritis in a subject, said array comprising, attached to its surface one, two, three, four, five, six, seven, eight or nine biomarkers selected from the group consisting of WIBG, TPM2, C17orf85, TCP10L, ZNF706, MGC17403, CCDC72, ELOF1 and GABARAPL2.

14. The array according to claim 13, said array comprising, attached to its surface said nine biomarkers.

15. The array according to claim 13, said array comprising, attached to its surface, the 4 biomarkers: WIBG, TPM2, ZNF706, and GABARAPL2.

16. The array according to claim 13, wherein the WIBG-protein is a peptide having an amino acid sequence comprising, or consisting of, a sequence selected from the group consisting of SEQ ID NO: 10, SEQ ID NO: 11, SEQ ID NO: 12 and SEQ ID NO: 13; the TPM2-protein is a peptide having an amino acid sequence comprising, or consisting of, a sequence selected from the group consisting of SEQ ID NO: 14 and SEQ ID NO: 15; the ZNF706-protein is a peptide having an amino acid sequence comprising, or consisting of, SEQ ID NO: 16, SEQ ID NO: 17, and SEQ ID NO: 18; and the GABARAPL2-protein is a peptide having an amino acid sequence comprising, or consisting of, SEQ ID NO: 19 and SEQ ID NO: 20.

17. The array according to claim 13 further comprising, attached to its surface, one or more additional biomarkers for detecting the presence of antinuclear antibodies and/or of anti-CCP antibodies.


SEQUENCE LISTING (20 sequences)

SEQ ID NO: 1 - protein, 204 aa, Homo sapiens
MEAAGSPAATETGKYIASTQRPDGTWRKQRRVKEGYVPQEEVPVYENKYVKFFKSKPELPPGLSPEATAPVTPSRPEGGEPGLSKTAKRNLKRKEKRRQQQEKGEAEALSRTLDKVSLEETAQLPSAPQGSRAAPTAASDQPDSAATTEKAKKIKNLKKKLRQVEELQQRIQAGEVSQPSKEQLEKLARRRALEEELEDLELGL

SEQ ID NO: 2 - protein, 284 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKYSTKEDKYEEEIKLLEEKLKEAETRAEFAERSVAKLEKTIDDLEDEVYAQKMKYKAISEELDNALNDITSL

SEQ ID NO: 3 - protein, 340 aa, Homo sapiens
MKYGNPNYGGMKGILSNSWKRRYHSRRIQRDVIKKRALIGDDVGLTSYKHRHSGLVNVPEEPIEEEEEEEEEEEEEEEEDQDMDADDRVVVEYHEELPALKQPRERSASRRSSASSSDSDEMDYDLELKMISTPSPKKSMKMTMYADEVESQLKNIRNSMRADSVSSSNIKNRIGNKLPPEKFADVRHLLDEKRQHSRPRPPVSSTKSDIRQRLGKRPHSPEKAFSSNPVVRREPSSDVHSRLGVPRQDSKGLYADTREKKSGNLWTRLGSAPKTKEKNTKKVDHRAPGAEEDDSELQRAWGALIKEKEQSRQKKSRLDNLPSLQIEVSRESSSGSEAES

SEQ ID NO: 4 - protein, 215 aa, Homo sapiens
MLAGQLEARDPKEGTHPEDPCPGAGAVMEKTAVAAEVLTEDCNTGEMPPLQQQIIRLHQELGRQKSLWADVHGKLRSHIDALREQNMELREKLRALQLQRWKARKKSAASPHAGQESHTLALEPAFGKISPLSADEETIPKYAGHKNQSATLLGQRSSSNNSAPPKPMSLKIERISSWKTPPQENRDKNLSRRRQDRRATPTGRPTPCAERRGGV

SEQ ID NO: 5 - protein, 76 aa, Homo sapiens
MARGQQKIQSQQKNAKKQAGQKKKQGHDQKAAAKAALIYTCTVCRTQMPDPKTFKQHFESKHPKTPLPPELADVQA

SEQ ID NO: 6 - protein, 351 aa, Homo sapiens
MSDKNQIAARASLIEQLMSKRNFEDLGNHLTELETIYVTKEHLQETDVVRAVYRVLKNCPSVALKKKAKCLLSKWKAVYKQTHSKARNSPKLFPVRGNKEENSGPSHDPSQNETLGICSSNSLSSQDVAKLSEMIVPENRAIQLKPKEEHFGDGDPESTGKRSSELLDPTTPMRTKCIELLYAALTSSSTDQPKADLWQNFAREIEEHVFTLYSKNIKKYKTCIRSKVANLKNPRNSHLQQNLLSGTTSPREFAEMTVMEMANKELKQLRASYTESCIQEHYLPQVIDGTQTNKIKCRRCEKYNCKVTVIDRGTLFLPSWVRNSNPDEQMMTYVICNECGEQWYHSKWVCW

SEQ ID NO: 7 - protein, 64 aa, Homo sapiens
MSGREGGKKKPLKQPKKQAKEMDEEDKAFKQKQKEEQKKLEELKAKAAGKGPLATGGIKKSGKK

SEQ ID NO: 8 - protein, 83 aa, Homo sapiens
MGRRKSKRKPPPKKKMTGTLETQFTCPFCNHEKSCDVKMDRARNTGVISCTVCLEEFQTPITYLSEPVDVYSDWIDACEAANQ

SEQ ID NO: 9 - protein, 117 aa, Homo sapiens
MKWMFKEDHSLEHRCVESAKIRAKYPDRVPVIVEKVSGSQIVDIDKRKYLVPSDITVAQFMWIIRKRIQLPSEKAIFLFVDKTVPQSSLTMGQLYEKEKDEDGFLYVAYSGENTFGF

SEQ ID NO: 10 - protein, 15 aa, Homo sapiens
MEAAGSPAATETGKY

SEQ ID NO: 11 - protein, 15 aa, Homo sapiens
AATETGKYIASTQRP

SEQ ID NO: 12 - protein, 15 aa, Homo sapiens
IASTQRPDGTWRKQR

SEQ ID NO: 13 - protein, 15 aa, Homo sapiens
KKKLRQVEELQQRIQ

SEQ ID NO: 14 - protein, 15 aa, Homo sapiens
QKMKYKAISEELDNA

SEQ ID NO: 15 - protein, 15 aa, Homo sapiens
ISEELDNALNDITSL

SEQ ID NO: 16 - protein, 15 aa, Homo sapiens
MARGQQKIQSQQKNA

SEQ ID NO: 17 - protein, 15 aa, Homo sapiens
IQSQQKNAKKQAGQK

SEQ ID NO: 18 - protein, 15 aa, Homo sapiens
KKQAGQKKKQGHDQK

SEQ ID NO: 19 - protein, 15 aa, Homo sapiens
MKWMFKEDHSLEHRC

SEQ ID NO: 20 - protein, 15 aa, Homo sapiens
GFLYVAYSGENTFGF